(19) United States
(12) Patent Application Publication
(10) Pub. No.: US 2008/0119895 A1
(43) Pub. Date: May 22, 2008

(54) Locking bone plate with bushing anti-rotation feature

(76) Inventor: Thierry Manceau, Saint-Martin d'Heres (FR)

Correspondence Address: FAEGRE & BENSON LLP;PATENT DOCKETING, 2200 WELLS FARGO CENTER, 90 SOUTH SEVENTH STREET, MINNEAPOLIS, MN, 55402-3901, US

(21) Appl. No.: 11/986,147

(22) Filed: Nov. 20, 2007

(30) Foreign Application Priority Data
Nov. 20, 2006 (FR) 0610141

(51) Int. Cl. A61B 17/58; A61B 17/56

(52) U.S. Cl. 606/280; 606/301

(57) ABSTRACT

A locking plate intended to be implanted at a bone site and a method for producing and implanting this device. At least one expandable bushing is engaged with a through-hole in the plate. The bushing includes an exterior surface with at least one recess and a passageway including a threaded interior surface. The expandable bushing is initially in a first configuration that permits poly-axial rotation of the bushing within the through-hole. An elongated anchoring member is provided with a distal portion and a proximal portion including a head portion with threads complementary to the threads on the interior surface of the expandable bushing. The proximal portion expands the bushing to form a friction lock between the bushing and the plate in a selected polyaxial position in a second configuration. At least one discrete blocking member is fixedly engaged with the body portion of the plate and extends into the through-hole to engage with the recess on the expandable bushing. The blocking member inhibiting rotation of the expandable bushing relative to the through-hole.

This application claims priority to French application no. 0610141, titled DISPOSITIF PROTHETIQUE OU D'OSTEOSYNTHESE A OLIVE FENDUE, filed on Nov. 20, 2006.

### FIELD OF THE INVENTION

The present invention is directed to an improved bone plate with an expandable bushing, in which the rotation of the bushing about its main axis is blocked before expansion, and to a method of making and implanting such a device.

### BACKGROUND OF THE INVENTION

Bone plates allow fractures of the anatomical neck of the humerus or of the end of the humerus situated nearer the elbow and also fractures of the upper end of the tibia, or even other epiphyseal lesions of long bones to be minimised.

WO-A-2003/007832 discloses a plate that is fixed to the bone by screws which each comprise, on the one hand, a threaded rod for anchoring to the bone, inserted through a through-hole in the plate, and, on the other hand, a threaded head for immobilising the screw relative to the plate, screwed into the wall of the hole, which is threaded in a complementary manner. In practice, the anchoring direction of the screws is not always aligned with the axis of revolution of the threaded wall of the hole in the plate and, as a result, it is necessary to interpose, between the head of the screw and the wall of the hole, a bushing for accommodating the misalignment between the axis of the screw and the axis of the hole. The exterior of the bushing is shaped in such a way that, as long as the head of the screw is not screwed into the bushing, the bushing is movable against a smooth wall of the hole like a ball joint, whereas, when the head of the screw is progressively screwed into the bushing, the bushing deforms so as to push firmly against the wall of the hole until it rigidly connects the screw and the plate by wedging. In practice, in order to be deformable, the bushing is split completely apart in such a way that the edges of the corresponding slot are spaced from one another when the head of the screw is screwed. An example of this is given in DE-U-200 22 673.

In use, such bushings are difficult to handle: when the head is screwed into the bushing, it is difficult for the internal thread of the bushing to engage with the thread of the screw head since the bushing tends to turn round on itself in the hole in the plate due to the fact that the connection between the bushing and the wall of the hole is still movable. A surgeon thus has difficulties in effectively screwing the head of the screw into the bushing and this complicates surgical procedures and increases intervention time.

To overcome this drawback, it is proposed that, when the head of the screw is not yet engaged in the bushing, the rotation of the bushing about the axis of its orifice be blocked relative to the hole in the prosthetic or osteosynthesis body, using an enlargement projecting from the outer face of the bushing which is received in a hollow groove in the wall of the hole. This enlargement abuts into one or the other of the ends of the groove when the bushing tends to rotate about the axis of its orifice, which rotationally immobilises the bushing about the axis, without preventing it from tilting within the hole in order to accommodate any possible misalignment between the axis of the hole and the anchoring direction of the screw selected by the surgeon. An example of this type of expandable bushing with a local enlargement on its outer face is shown in FIG. 15 to 20 of document US-A-2005/0154392.

In practice, forming a rotation-blocking enlargement of this type causes difficulties in production since the presence of the enlargement must not adversely affect the geometric features of the rest of the outer face of the bushing, for example its spherical shape, without which the bushing tends to become wedged in the hole in undesirable positions. In order to obtain an acceptable level of reliability, production costs are therefore high.

Another solution which blocks the bushing in rotation is proposed by US-A-2005/085913, which discusses, without illustrating, a member or a projecting enlargement located inside the hole for receiving the bushing and provided for extending into the slot of the bushing. US-A-2005/085913, however, does not provide any corresponding details regarding production thereof and thus does not provide an actual practical and cost-effective solution.

### BRIEF SUMMARY OF THE INVENTION

The present invention is directed to an improved bone plate with an expandable bushing, in which the rotation of the bushing about its main axis is blocked before expansion, which is simple to produce, effective, and inexpensive.

The locking plate includes a plate with a body portion and at least one through-hole. At least one expandable bushing is engaged with the body portion. The bushing includes an exterior surface with at least one recess and a passageway including a threaded interior surface. The expandable bushing is initially in a first configuration that permits poly-axial rotation of the bushing within the through-hole. An elongated anchoring member is provided with a distal portion and a proximal portion including a head portion with threads complementary to the threads on the interior surface of the expandable bushing. The proximal portion expands the bushing to form a friction lock between the bushing and the plate in a selected polyaxial position in a second configuration. At least one discrete blocking member is fixedly engaged with the body portion of the plate and extends into the through-hole to engage with the recess on the expandable bushing. The blocking member inhibiting rotation of the expandable bushing relative to the through-hole.

In one embodiment, the recess is a slot extending completely through a wall of the expandable bushing. Radial expansion of the expandable bushing from the first configuration to the second configuration is typically plastic and/or elastic deformation of the expandable bushing. The exterior surface of the expandable bushing and walls of the through-hole are preferably substantially spherical in a complementary manner, except where the blocking member engages with the recess.

The distal portion of the anchoring member can be smooth, or include threads adapted to engage with the bone. The threads on the head portion of the anchoring member and the threads on the interior surface of the bushing can be tapered or cylindrical. In one embodiment, the head portion includes an unthreaded tapered surface that expands the expandable bushing to the second configuration.

The blocking member is preferably a discrete component fixedly engaged with a recess in the body portion of the plate, such as for example a cylinder that engages with a hole in the body portion of the plate. A single blocking member can extend into a plurality of through-holes to simultaneously engage with the recesses of a plurality of expandable bushings. In some embodiments, the recess in the body portion of the plate includes a central axis that is parallel to, or at an angle with respect to, a central axis of the through-hole.

The present invention is also directed to a method of engaging a locking plate with a bone. The method includes positioning the locking plate against a bone. An expandable bushing is located in at least one through-hole in a body portion of the locking plate. An elongated anchoring member is inserted through a passageway in the expandable bushing. The bushing is poly-axially rotated within the through-hole to a desired angle. The distal portion of the anchoring member is inserted into the bone. At least one blocking member is engaged with the body portion of the plate so that the blocking member extends into the through-hole to engage with a recess on the expandable bushing and block rotation of the expandable bushing relative to the through-hole. Threads on a head portion of the anchoring member are engaged with a threaded interior surface of the passageway on the bushing to expand the bushing from a first configuration that permits poly-axial rotation of the bushing within the through-hole to a second configuration that comprises a friction lock between the bushing and the plate.

The bushing can be expanded plastically or elastically. In one embodiment, threads on a distal portion of the anchoring member engage with the bone. In another embodiment, tapered threads on one or more of the head portion and the threads on the interior surface of the bushing radially expand the bushing from the first configuration to the second configuration. In another embodiment, a tapered surface on a proximal portion of the head portion radially expands the bushing from the first configuration to the second configuration.

The blocking member is preferably a discrete component fixedly engaged with a recess in the body portion of the plate. For example, a cylindrical blocking member is inserted into a cylindrical hole on the body portion of the bone plate. A single blocking member can optionally extend into a plurality of through-holes to simultaneously engage with the recesses of a plurality of expandable bushings.

### BRIEF DESCRIPTION OF THE SEVERAL VIEWS OF THE DRAWING

A better understanding of the invention will be facilitated by reading the following description, given by way of example only with reference to the drawings, in which:

FIG. 1 is a perspective view of the upper end of a humerus including a locking bone in accordance with an embodiment of the present invention.

FIG. 2 is a partial cross-section along the plane II in FIG. 1 showing in an exploded manner the humeral plate as well as the blocking members, a bushing, and an anchoring member of the device.

FIG. 3 is a view directed along the arrow III in FIG. 2 showing one of the blocking members, the bushing and a portion of the plate of the device.

FIG. 4 is a perspective view of an alternate locking plate in accordance with an embodiment of the present invention.

FIG. 5 is a partial cross-section along the plane V of FIG. 4 showing in an exploded manner the components of FIG. 4 as well as a bushing and an anchoring member of the device.

FIG. 6 is cross-sectional view of an alternate bushing and anchoring member in according with an embodiment of the present invention.

FIG. 7 is a top view of an alternate bushing and a portion of the plate of the device in accordance with an embodiment of the present invention.

### DETAILED DESCRIPTION OF THE INVENTION

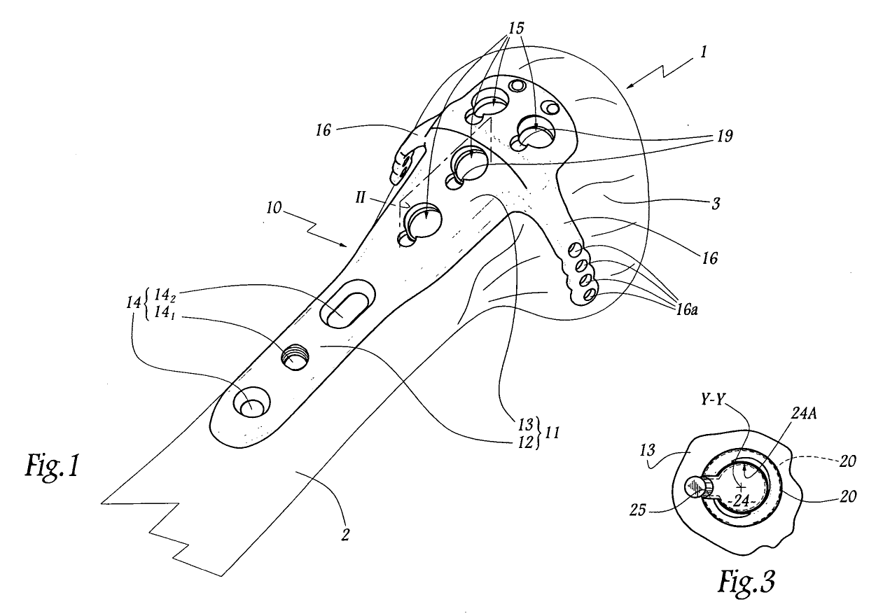

In FIG. 1, the upper end portion of a humerus is schematically shown, the diaphysis and the epiphysis of which are referenced with the numerals 2 and 3 respectively. For the sake of convenience the following description will refer to the humerus in its anatomical position for a patient standing erect, in such a way that the terms “upper” and “high” designate an upwards direction in FIGS. 1 to 3, while the terms “lower” and “low” designate the opposite direction.

On the humerus 1 is fitted a humeral plate 10 comprising a main elongate body 11 extending in the longitudinal direction of the humerus, both at the diaphysis 2 thereof and at the epiphysis 3 thereof. The body 11 thus includes a diaphyseal portion 12 and an epiphyseal portion 13 located at the diaphysis and the epiphysis respectively of the humerus.

A plurality of holes 14 pass through the thickness of the portion 12 and open onto the humeral diaphysis 2, including a threaded hole 14₁ and a hole 14₂ with an oblong cross-section. The holes 14 are not described in further detail, it being noted that their number and/or their geometry do not limit the invention in any way.

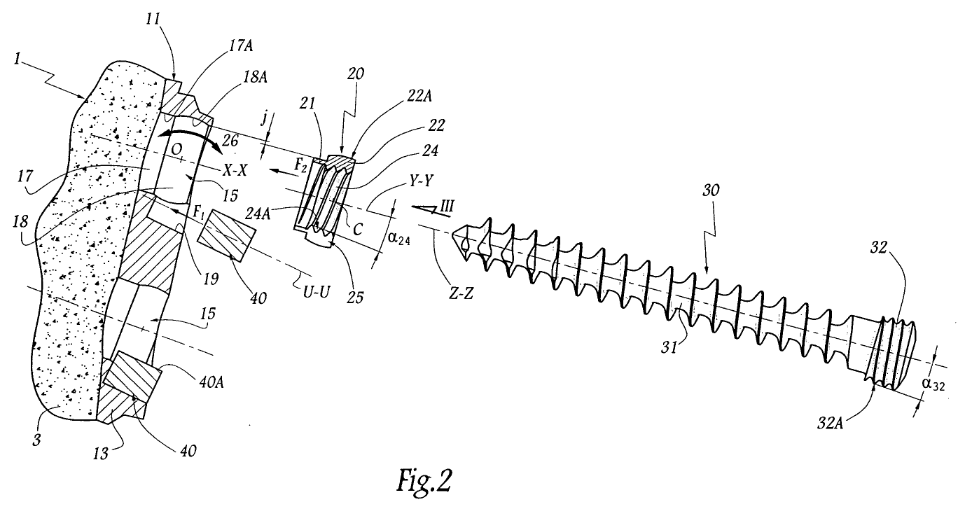

In the illustrated embodiment, four holes 15 pass through the thickness of the epiphyseal portion 13 and open onto the humeral epiphysis 3 and are identical to one another. The two lowest holes 15, seen in cross-section in FIG. 2, are disposed one behind the other in the longitudinal direction of the body 11, while the two remaining holes are disposed symmetrically on both sides of the centre plane of the body, which corresponds to the plane of cross-section II.

The exemplary humeral plate 10 also comprises two lateral projections 16 which extend on both sides of the epiphyseal portion 13 each in opposite directions, transverse to the longitudinal direction of the body. In cross-section, namely in a cross-sectional plane substantially perpendicular to the plane II, the arms 16 and the portion 13 are generally C-shaped and are sized to embrace the epiphysis 3 in order to improve the stability of the plate 10 on the humerus 1.

In order to fix the plate 10 to the humerus 1, a plurality of structures for anchoring to the bone may be used by each being inserted into the holes 14 of the diaphyseal portion 12, into the holes 16a provided at the free end of each lateral projection 16, and into the holes 15 of the epiphyseal portion 13. Only the anchoring structure associated with the holes 15 will be described in detail in the following, it being understood that the configuration of the plate 10 and the number and location of the holes 14, 15, 16a do not limit the invention.

As shown in more detail in FIGS. 2 and 3, each through-hole 15 comprises, in sequence, on the one hand, on the side of the body 11 to be turned towards the humerus 1, a cylindrical portion 17 with a circular base and having a longitudinal axis X-X and, on the other hand, on the opposite side, a spherical portion 18 with its centre at a point O on the axis X-X. The spherical portion 18 substantially corresponds, along axis X-X, to the median zone of the geometrical sphere to which the portion 18 belongs, in such a way that the point O is located inside portion 18.

The axes X-X of each hole 15 are not necessarily parallel to one another, as shown by the two holes seen in cross-section in FIG. 2. Each hole 15 is adapted to receive both a generally tubular bushing 20 with a longitudinal axis Y-Y, and an anchoring member 30 for anchoring to the bone with a longitudinal axis Z-Z.

The portion of the bushing 20 closes to the humerus 1 comprises an annular portion 21 with a circular base with an axis Y-Y and, at the opposite side, a ring-shaped portion 22 with a substantially spherical outer face 22A with its centre at a point C on the axis Y-Y. The annular portion 21 and the substantially spherical outer face 22A preferably do not contain any protrusions or other structures. Consequently, the bushing 20 can be manufactured using conventional machining operations.

The outer diameter of the annular portion 21 is strictly less than the inner diameter of the portion 17 of the hole 15, whereas the outer diameter of the portion 22 is substantially equal to the inner diameter of the portion 18 of the hole. In this way, when the bushing 20 is received in the hole 15, a not-insignificant radial clearance j is present between the portions 21 and 17, whereas the spherical face 22A of the portion 22 and the spherical wall 18A of the portion 18 are juxtaposed in a complementary manner, the points 0 and C thus substantially coinciding.

At its interior, the bushing 20 delimits a through-orifice 24 centred on the axis Y-Y and threaded over a portion of its length along the axis. In the illustrated embodiment, the bushing includes slot 25 on a portion of its periphery, which extends over the entire bushing in the direction of the axis Y-Y and which passes completely through the tubular wall of the bushing in such a way that the slot radially connects the outer face of the bushing and the inner orifice 24 thereof. The slot 25 thus imparts a shape in the general form of a C to the bushing when viewed in cross-section, as well as when viewed along the axis Y-Y, as shown in FIG. 3.

Along its length, the anchoring member 30 comprises, on the one hand, a distal threaded rod 31 to be screwed into the thickness of the bone of the humeral epiphysis 3 and, on the other hand, a proximal threaded head 32 to be screwed into the inner orifice 24 of the bushing 20. The screw head is adapted to allow the anchoring member 30 to be rotationally driven about its axis Z-Z, both to screw the rod 31 into the humeral bone and to screw its head 32 into the orifice 24 of the bushing. For this purpose, the head 32 has, for example, on the proximal side thereof, a cavity (not shown in the figures) with a hexagonal or similar profile, which allows the anchoring member 30 to be rotationally-driven by using an appropriate tool.

In one embodiment, the outer threaded face 32A of the head 32 is in the form of truncated-cone-shaped casing with an axis Z-Z converging towards the axis as it approaches the rod 31. The wall 24A of the threaded orifice 24 of the bushing 20 is also optionally in the form of truncated-cone-shaped casing with an axis Y-Y, converging towards the axis as it approaches the distal side of the bushing. The truncated-cone shapes of the outer face 32A and the wall 24A are substantially complementary, the angles at the apexes α₃₂ and α₂₄ of these two truncated-cone shapes being substantially equal. In another embodiment, only one of the threaded face 32A or the wall 24A comprise a truncated cone and the other comprises a cylindrical configuration without a taper.

In addition, the outer diameter of the distal end of the head 32 is substantially equal to the inner diameter of the proximal end of the orifice 24 in such a way that, as the head 32 is screwed into the bushing 20, the inner diameter of the orifice 24 increases until the inner diameter of the proximal end of the orifice is substantially equal to the outer diameter of the proximal end of the head 32 when the head is completely screwed into the bushing. Increasing the inner diameter of the orifice 24 is enabled by the fact that the bushing 20 is split. When the head 32 is screwed into the orifice 24, the edges of the slot 25 move away from one another, and thus allow the bushing to pass from a first configuration, shown in dashed lines in FIG. 3 and corresponding to a rest state of the bushing 20 in the hole 15, to a second configuration in which it is radially deformed towards the exterior in relation to the axis Y-Y, shown in a solid line in FIG. 3. The bushing 20 may be constructed of a material that permits plastic or elastic deformation.

FIG. 7 illustrates an alternate bushing 120 located in a hole 15 of the epiphyseal portion 13. Rather than slot 25 illustrated in FIG. 3, the bushing 120 includes a recess 122 that engages with blocking member 40. In the illustrated embodiment, slit 124 is provided to permit the bushing 120 to expand radially when engaged with an anchoring member. In an alternate embodiment, the slit 124 is located adjacent to the recess 122. In another alternate embodiment, the bushing 120 is constructed from a deformable or expandable material, such as for example, a biocompatible polymer that plastically deforms when engaged with an anchor member 30, without the need for the slit 124 or the slot 25.

In order to block the bushing 20 from rotating about the axis Y-Y in the hole 15 when it is in its first configuration, a member 40 is inserted fixedly into the body 11 of the plate 10. In the illustrated embodiment, the member is in the form of a cylinder with a circular base and having a central longitudinal axis U-U, formed in particular from a metal similar to that of the body of the plate 11. The blocking member 40 is joined to the plate 10 by being received and immobilised, in particular by welding, in a complementary recess 19 formed, for example by machining, in the body of the plate 11, at a peripheral portion of the hole 15. In practice, the recess 19 is smaller than the hole 15 in that the diameter thereof is smaller than that of the hole. The blocking member 40 may be other shapes, such as for example with a narrowing configuration that tapers in the direction of the centre of the hole 15 that they occupy in part, so as not to obstruct the partial closure of the slot during introduction of the bushing into the hole, while being adjusted closest to the edge of this slot once the bushing is received in this hole.

The central longitudinal axis of the recess 19 may be parallel to or, as for example shown in the figures, slightly inclined so as to converge towards the humerus, preferably at an angle of about 15° or less with respect to the axis X-X of the hole, opens radially into the hole 15. In other words, the hole 15 and the recess 19 are not respectively closed over all of their periphery, but respective portions of the peripheries are provided so as to communicate with one another, in particular in the plane of FIG. 2. The recess 19 is thus in the form of a portion of a cylinder. In this way, when the member 40 is received in the recess 19, as illustrated for the hole 15 shown in the lower portion of FIG. 2, and as illustrated in FIG. 3, the member occupies all of the internal volume of the recess, while a peripheral portion 40A of the member occupies a portion of the interior of the corresponding hole 15. The portion 40A is provided to extend, in a direction peripheral to the axis Y-Y, between the edges of the slot 25 when the bushing is received in the hole 15, as shown in FIG. 3.

Producing and implanting the plate 10, the bushing 20, the anchoring member 30 and the member 40, will now be discussed in more detail.

It is proposed that the four members 40, each associated with the four holes 15, are initially joined to the plate 10 by welding each of the members in the corresponding recess 19 previously formed in the body of the plate 11, as shown by the arrow F₁ in FIG. 2. The bushings 20 are then received in their associated hole 15, each of the slots 25 thereof being positioned in an angled manner such that the peripheral portion 40A of each member 40 is received between the edges of the slot, as shown by arrow F₂.

In practice, fitting each bushing 20 requires that the externally spherical portion 22 is gently radially compressed towards the interior by bringing the edges of its slot 25 slightly closer together until the maximum outer diameter of the bushing is less than the proximal diameter of the hole 15. The whole of the bushing may then be axially inserted into the hole, the portion of the member 40A being received in the slot 25. If necessary, the slot 25 may be partially closed again until the edges thereof contact the lateral wall of the portion of the member 40A. The cylindrical shape of the lateral wall has been found to be practical as it does not obstruct the partial closure of the slot 25 and it can even guide the insertion of the bushing into the hole by sliding contact of the edges of the slot thereof along the lateral wall.

The surgeon then fits the plate 10 along the humerus 1, the portion 13 of the body 11 and the lateral projections 16 embracing the epiphysis 3 thereof, as shown in FIG. 1.

The anchoring member 30 is thus axially inserted into each hole 15 by rotationally driving the anchoring member about its axis Z-Z in such a way that the rod 31 thereof penetrates into the bone matter of the epiphysis 3 in order to be securely anchored therein. The anchoring member 30 is inserted and screwed in while the bushing 20 is received in the hole 15, the anchoring member 30 passing through the orifice 24 thereof. In this configuration, the spherical face 22A of the bushing slides freely against the spherical wall 18A of the hole 15 in order to adjust for misalignment between the axes X-X and Z-Z if the surgeon inserts the anchoring member 30 in an inclined longitudinal direction relative to the axis of the hole, in particular as a function of the state of the bone matter at his disposal. The movable connection between the bushing and the wall of the hole 15 is similar to that of a ball joint with, however, its freedom of movement restricted by the presence of the member 40.

The member 40A prevents the bushing 20 from turning on itself so that the passageway 24 is inaccessible. In other words, in order to accommodate any potential misalignment of the axes X-X and Z-Z, the bushing 20 is able to pivot inside the hole 15 about an axis which is substantially perpendicular to the axis X-X and passing through the point O, by sliding contact of the face 22A against the wall 18A, as shown by the arrow 26 in FIG. 2. The pivot range is limited by the annular portion 21 being brought into abutment against the wall 17A of the portion of the hole 17. The maximum pivot range of the bushing is directly related to the aforementioned clearance j and is in the range of about 20°.

When the head 32 is axially directly adjacent to the bushing 20 and when the surgeon continues to rotationally drive the anchoring member 30 about its axis Z-Z, the thread of the outer face 32A of the head 32 engages simply and easily in the thread of the orifice 24, causing the bushing to pass from its first to its second configuration. Initially engaging the thread of the anchoring member head in the internal thread of the bushing is facilitated by the fact that the rotation of the bushing 20 about its axis Y-Y is immobilised by the member 40.

As the head 32 is screwed into the threaded orifice 24, the outer face 22A of the bushing 20 is pressed against the wall 18A of the hole 15 until the bushing is wedged inside the hole, thus rigidly connecting the bushing, and therefore the anchoring member 30, to the plate 10.

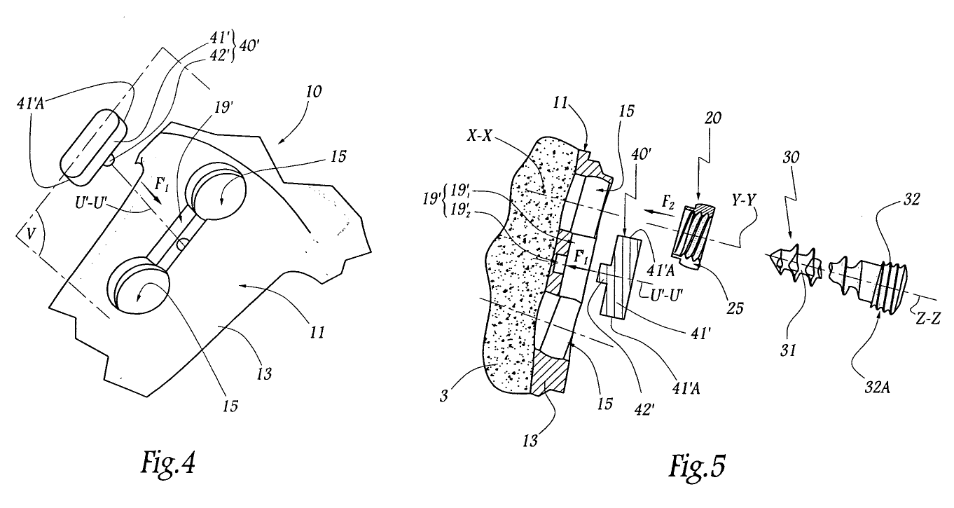

FIGS. 4 and 5 show a variation of the osteosynthesis device in FIGS. 1 to 3 is shown, the components common to the two embodiments have the same reference numerals. The variant in FIGS. 4 and 5 is distinguished from the device in FIGS. 1 to 3 basically through the shape of at least one of the members 40′ for rotationally blocking the bushings 20 in the holes 15. More precisely, rather than associating one of the cylindrical members 40 of the device in FIGS. 1 to 3 with each hole 15, the same member 40′ allows rotational locking of two bushings 20 received in two adjacent holes 15, for example in the two lowest holes 15 of the humeral plate 10, as shown in FIGS. 4 and 5. Of course, a member of the same type as the member 40′ described hereinafter may be used, in a non-illustrated variant, to block the bushings 20 received in the two holes 15 located in the upper portion of the body of the plate 11 on both sides of the centre plane of the body.

As shown in FIGS. 4 and 5, the member 40′ comprises a generally parallelepiped proximal portion 41′ and a generally cylindrical distal portion 42′ with a circular base, centred on an axis U′-U′ perpendicular to the longitudinal dimension of the portion 41′. In the layout of FIG. 5, the member 40′ is thus generally T-shaped.

In order to receive and immobilise the member 40′, the plate body 11 delimits a recess 19′ which includes, on the proximal side of the plate body, an elongate portion 19′₁ which extends lengthwise between the two holes 15 in the plane passing through the axes X-X of the holes, generally in a direction radial to the holes. The longitudinal ends of the portion of recess 19′₁ open respectively into the two holes 15.

On the distal side of the plate body 11, the recess 19′ includes a cylindrical portion 19′₂ opening onto the portion of the recess 19′₁ in such a way that the portions of the recess 19′₁ and 19′₂ are respectively complementary to the proximal portion 41′ and the distal portion 42′ of the member 40′.

In order to insert the member 40′ into the recess 19′, the distal portion 42 is coaxially inserted into the portion of the recess 19′₂ as indicated by the arrow F′₁, while the proximal portion 41′ is inserted in an adjusted manner into the portion of the recess 19′₂, thus preventing the member 40′ from turning about the axis U′-U′. The longitudinal ends 41′A of the proximal portion 41′ thus occupy a portion of each of the holes 15 in the same way as the peripheral portion 40A of the member 40 occupies a portion of the hole 15 in the embodiment in FIGS. 1 to 3. Advantageously the longitudinal ends 41′A are shaped as cylinder portions, the respective longitudinal axes of which are shown in the layout in FIG. 5 so as to facilitate the fitting of the bushings 20 into the holes 15.

The manufacture and use of the variant in FIGS. 4 and 5 are similar to those of the device envisaged in FIGS. 1 to 3. Once the member 40′ has been placed and interlocked in the recess 19′, the bushings 20 are each introduced into one of the holes 15, with the ends 41′A of the member 40′ received between the edges of the slot 25 of each bushing, as indicated by the arrow F₂. The screws 30 are then introduced axially into each hole 15, as described above with regard to the embodiment of FIGS. 1 to 3.

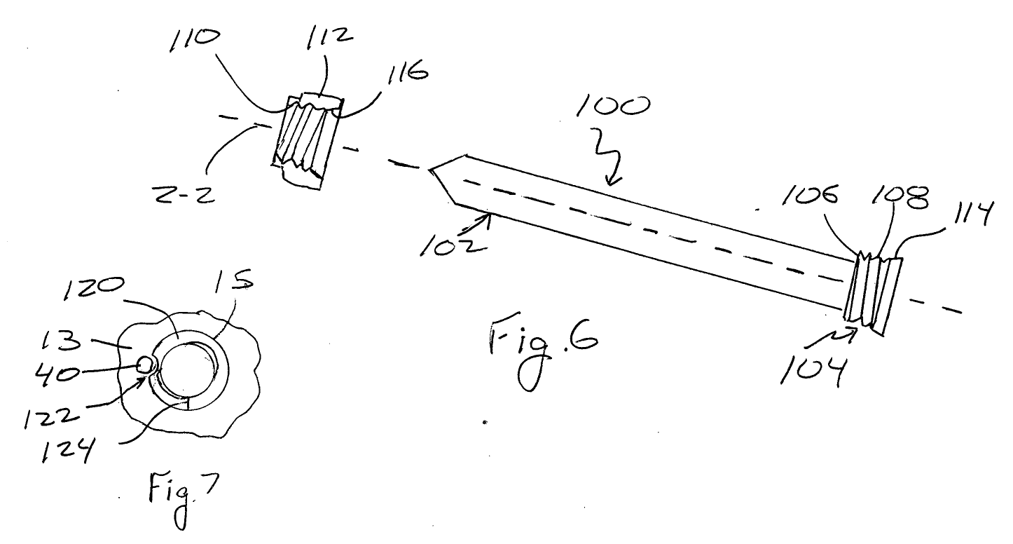

FIG. 6 illustrates an alternate anchoring structure 100 in the form of a pin or nail on which the distal portion 102 is not threaded. The surgeon will typically drill a hole in the bone to receive the unthreaded distal portion 102 of the anchoring structure 100. Proximal portion 104 comprises a head 106 with threads 108 complementary to threads 110 on the bushing 112. In the illustrated embodiment, the threads 108 and 110 are not tapered. Rather, sloped surface 114 on the head 106 engages with surface 116 on the bushing 112. Once the surfaces 114 and 116 are engaged, further rotation of the anchoring member causes the bushing to expand radially relative to the axis Z-Z′.

The present expandable bushing and anti-rotation member can be used with a variety of other orthopaedic implants, such as for example a base plate for a glenoid implant. Examples of such base plates are illustrated in U.S. Pat. Nos. 6,969,406, 6,761,746, 5,702,447 and U.S. Patent Publication No. 2005/0278032, which are hereby incorporated by reference.

Patents and patent applications disclosed herein, including those cited in the Background of the Invention, are hereby incorporated by reference. Other embodiments of the invention are possible. Although the description above contains many specificities, these should not be construed as limiting the scope of the invention, but as merely providing illustrations of some of the presently preferred embodiments of this invention. Thus the scope of this invention should be determined by the appended claims and their legal equivalents. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims.

What is claimed is:

1. A locking plate for engagement with a bone, the apparatus comprising:
a plate including a body portion and an at least one through-hole;
an expandable bushing comprising an exterior surface comprising at least one recess and a passageway comprising a threaded interior surface, the expandable bushing comprising a first configuration that permits poly-axial rotation of the bushing within the through-hole;
an elongated anchoring member comprising a distal portion and a proximal portion comprising a head portion with threads complementary to the threads on the interior surface of the expandable bushing, the proximal portion expanding the bushing to form a friction lock between the bushing and the plate in a selected polyaxial position in a second configuration; and
at least one discrete blocking member fixedly engaged with the body portion of the plate and extending into the through-hole to engage with the recess on the expandable bushing, the blocking member inhibiting rotation of the expandable bushing relative to the through-hole.

2. The locking plate of claim 1 wherein the recess comprises a slot extending completely through a wall of the expandable bushing.

3. The locking plate of claim 1 wherein radial expansion of the expandable bushing from the first configuration to the second configuration comprises one of plastic or elastic deformation of the expandable bushing.

4. The locking plate of claim 1 wherein the exterior surface of the expandable bushing and walls of the through-hole are substantially spherical in a complementary manner, except where the blocking member engages with the recess.

5. The locking plate of claim 1 wherein the distal portion of the anchoring member comprises threads adapted to engage with the bone.

6. The locking plate of claim 1 wherein one or more of the threads on the head portion and the threads on the interior surface of the bushing comprise tapered threads.

7. The locking plate of claim 1 comprising a tapered surface on the proximal portion of the head portion, the tapered surface expanding the expandable bushing to the second configuration.

8. The locking plate of claim 1 wherein the blocking member comprises a discrete component fixedly engaged with a recess in the body portion of the plate.

9. The locking plate of claim 1 wherein the blocking member comprises a cylinder.

10. The locking plate of claim 1 wherein a single blocking member extends into a plurality of through-holes to simultaneously engage with the recesses of a plurality of expandable bushings.

11. The locking plate of claim 1 comprises a recess in the body portion of the plate sized to receive the blocking member, the recess comprising a central axis that is parallel to, or at an angle with respect to, a central axis of the through-hole.

12. A method of engaging a locking plate with a bone, the method comprising the steps of:
positioning the locking plate against a bone;
locating an expandable bushing in at least one through-hole in a body portion of the locking plate;
inserting an elongated anchoring member through a passageway in the expandable bushing;
poly-axially rotating the bushing within the through-hole and inserting the anchoring member into the bone;
engaging at least one blocking member with the body portion of the plate so that the blocking member extends into the through-hole to engage with a recess on the expandable bushing and blocks rotation of the expandable bushing relative to the through-hole; and
engaging threads on a head portion of the anchoring member with a threaded interior surface of the passageway on the bushing to expand the bushing from a first configuration that permits poly-axial rotation of the bushing within the through-hole to a second configuration that comprises a friction lock between the bushing and the plate.

13. The method of claim 12 comprising one of plastically or elastically deforming the expandable bushing from the first configuration to the second configuration.

14. The method of claim 12 comprising engaging threads on a distal portion of the anchoring member with the bone.

15. The method of claim 12 wherein tapered threads on one or more of the head portion and the threads on the interior surface of the bushing radially expand the bushing from the first configuration to the second configuration.

16. The method of claim 12 wherein a tapered surface on a proximal portion of the head portion radially expands the bushing from the first configuration to the second configuration.

17. The method of claim 12 wherein the blocking member comprises a discrete component fixedly engaged with a recess in the body portion of the plate.

18. The method of claim 12 comprising inserting a cylindrical blocking member into a cylindrical hole on the body portion of the bone plate.

19. The method of claim 12 comprising positioning a single blocking member to extend into a plurality of through-holes to simultaneously engage with the recesses of a plurality of expandable bushings.

20. The method of claim 12 comprises inserting the blocking member into a recess in the body portion of the plate, the recess comprising a central axis that is parallel to, or at an angle with respect to, a central axis of the through-hole.
